(19) United States
(12) Patent Application Publication
(10) Pub. No.: US 2026/0167983 A1
(43) Pub. Date: Jun. 18, 2026

(54) METHODS AND COMPOSITION FOR NOVEL DITERPENE SYNTHESIS INHIBITOR HERBICIDE RESISTANT RICE LINES

(71) Applicant: RiceTec, Inc., Alvin, TX (US)

(72) Inventors: Dario Bernacchi, Friendswood, TX (US); Caleb Knepper, League City, TX (US); Anuj Kumar, League City, TX (US)

(21) Appl. No.: 19/420,031

(22) Filed: Dec. 15, 2025

Related U.S. Application Data
(60) Provisional application No. 63/734,951, filed on Dec. 17, 2024.
(60) Provisional application No. 63/741,503, filed on Jan. 3, 2025.

(51) Int. Cl. C12N 15/82; A01N 43/80; A01P 13/02

(52) U.S. Cl. CPC C12N 15/8274; A01N 43/80; A01P 13/02

(57) ABSTRACT

Rice is described that is tolerant/resistant to high-dose applications of the diterpene biosynthesis inhibiting herbicide clomazone. Putative causal non-target site mutations were identified on chromosome 2 of rice. Use of the high clomazone dose tolerant rice lines for more effective weed control, as well as methods of producing tolerant/resistant rice are also disclosed.

### CROSS-REFERENCE TO RELATED APPLICATIONS

This application claims the benefit of priority to U.S. Provisional Patent Application Nos. 63/741,503 filed Jan. 3, 2025, and 63/734,951 filed Dec. 17, 2024, the disclosures of which is hereby incorporated by reference in their entireties.

### BACKGROUND

Mutant rice is disclosed that is resistant/tolerant to diterpene synthesis inhibitor herbicides, especially Clomazone, at a relatively high concentration of application, generated by random mutagenesis. Methods of weed control are disclosed using herbicide resistant/tolerant rice with these mutation lines as crops in fields. Methods to produce herbicide resistant/tolerant rice are also disclosed.

### Value of Rice Crop

Rice is an ancient agricultural crop and today is one of the principal food crops of the world. There are two cultivated species of rice: Oryza sativa L., the Asian rice, and Oryza glaberrima Steud., the African rice. The Asian species constitutes virtually all of the world's cultivated rice and is the species grown in the United States. Three major rice producing regions exist in the United States: the Mississippi Delta (Arkansas, Mississippi, northeast Louisiana, southeast Missouri), the Gulf Coast (southwest Louisiana, southeast Texas), and the Central Valley of California. Other countries, in particular in South America and the East, are major rice producers.

Rice is one of the few crops that can be grown in a shallow flood as it has specialized tissues allowing gas exchange through the stems between the roots and the atmosphere. Growth in a shallow flood, provides weed control resulting in the best yields and is the reason that rice is usually grown in heavy clay soils, or soils with an impermeable hard pan layer just below the soil surface. These soil types are usually either not suitable for other crops or at best, the crops yield poorly.

### Breeding Improved Rice Seed

The constant improvement of rice is imperative to provide necessary nutrition for a growing world population. A large portion of the world population consumes rice as their primary source of nutrition and crops must thrive in various environmental conditions including competing with weeds and attacks by unfavorable agents. Rice improvement is carried out through conventional breeding practices complemented by the application of other established and new breeding techniques. Though appearing straightforward to those outside this discipline, crop improvement requires keen scientific and artistic skill and, in certain implementations, results can be unpredictable, as is the case of random mutagenesis trait discovery. Although specific breeding objectives vary somewhat in the different rice producing regions of the world, increasing yield is a primary objective in all programs.

Traditional plant breeding begins with the analysis and definition of strengths and weaknesses of cultivars in existence, that are adapted to the production region, followed by the establishment of program goals, to improve areas of weakness to produce new cultivars. Specific breeding objectives include combining in a single cultivar an improved combination of desirable traits from the parental sources. Desirable traits may be introduced by spontaneous or induced mutations and also through the utilization of unadapted germplasm. Desirable traits include higher yield, resistance to environmental stress, diseases and insects, better stems and roots, tolerance to low temperatures, better agronomic characteristics, nutritional value and grain quality.

For example, the breeder initially selects and crosses two or more parental lines, followed by selection for desired traits among the many new genetic combinations. The breeder can theoretically generate millions of new and different genetic combinations via crossing, to be advanced by inbreeding, and tested for performance and productivity.

Pedigree breeding is used commonly for the improvement of self-pollinating crops such as rice. For example, two parents which possess favorable, complementary traits are crossed to produce an F1 generation. One or both parents may themselves represent an F1 from a previous cross. Subsequently a segregating population is produced, by growing the seeds resulting from selfing one or several F1s if the two parents are pure lines, or by directly growing the seed resulting from the initial cross if at least one of the parents is an F1. Selection of the best individual genomes may begin in the first segregating population or F2; then, beginning in the F3, the best individuals in the best families are selected. “Best” is defined according to the goals of a particular breeding program e.g., to increase yield, resist diseases. Overall, a multifactorial approach is used to define “best” because of genetic interactions. A desirable gene in one genetic background may differ in a different background. In addition, introduction of the gene may disrupt other favorable genetic characteristics. Replicated testing of families can begin in the F4 generation to improve the effectiveness of selection for traits with low heritability. At an advanced stage of inbreeding (i.e., F6 and F7), the best lines or mixtures of phenotypically similar lines are tested for potential release as new parental lines.

Promising advanced breeding lines are thoroughly tested and compared to appropriate standards in environments representative of the commercial target area(s) for at least three or more years. The best lines are candidates for new commercial varieties or parents of hybrids; those still deficient in a few traits may be used as parents to produce new populations for further selection.

These processes, which lead to the final step of marketing and distribution, usually take from 8 to 12 years from the time the first cross is made and may rely on the development of improved breeding lines as precursors. Therefore, development of new cultivars is not only a time-consuming process, but requires precise forward planning, efficient use of resources, and a minimum of changes in direction. The results include novel genetic combinations not found in nature.

Hybrid breeding, also used in rice, as well as in other crops leverages the heterotic potential between inbred lines that have been selected for specific combining ability, whereby the productivity and performance of the progeny of their cross, shows phenotypic values far superior to those of either parent. In contrast to pedigree breeding for inbred lines of high phenotypic performance, Hybrid Breeding involves pedigree breeding with separate Male and Female heterotic groups for, respectively, male and female trait attributes, and general combining ability, followed by a stage of hybrid breeding whereby specific female male line combination are tested for specific combining ability.

Breeding by using crossing and selfing, for lines or hybrids of enhanced performance, does not imply direct control of modifications at the cellular level. However, that type of control may be achieved in part using recombinant genetic techniques and techniques capable of inducing modification in a plant or cell genome.

### Trait Discovery and Breeding for Improved Rice Seed

In addition to adaptability and performance, generally controlled by a multiplicity of genes of small effect and therefore requiring progressive breeding program application for steady genetic gain, many critical traits of commercial interest are controlled by simpler genetic factors, such as disease, insect, grain quality or herbicide resistances amongst others. These traits are often of great economic importance in that they add value or enable the high productivity genetic potential of the best selected products advanced by breeders.

The genetic variants controlling these traits may or may not be present in the elite breeding germplasm, or even in cross-compatible unadapted germplasm. To access these traits, the breeder must follow either of two approaches.

The first approach contemplates accessing and integrating rice germplasm that may have that trait but it is not adapted to the region in question, and is not part of the breeding pools in utilization. This approach requires prolonged breeding and special selection approaches, such as backcross breeding technique whereby the breeder achieves selection of targeted trait, retention of the novel trait genetic factors, and selection against other undesirable characteristics of the donor unadapted parent. Backcross breeding has been used to transfer genes for a highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The process is used to recover all of the beneficial characteristics of the recurrent parent with the addition of the new trait provided by the donor parent.

The second approach involves de-novo generation of genetic variance for that trait by induction of mutations, through traditional random approaches, or through the application of modern site directed mutagenesis techniques such as Gene Editing.

Traditional random mutagenesis techniques induce small changes in the DNA sequence that may result in a new trait of interest, as may occur spontaneously in nature. Commonly, Ethyl methanesulfonate (EMS) or sodium azide plus N-methyl-N-nitrosourea (MNU) are used as mutagenic agents. These chemicals randomly induce single base changes in DNA, usually of G and C changed to A and T. Overall effects are unpredictable. Most of these changes have no effect on the crop, because they fall either outside the gene coding regions or do not change the amino acid sequence of the gene product. However, some produce new traits or incorporate new DNA changes into previous lines. In random mutagenesis, the breeder has no direct control of mutation sites in the DNA sequence. The identification of useful changes is usually due to the random possibility that an effective mutation will be induced, and that the breeder will recognize the phenotypic effects of the change and will be able to select rice having that mutation for production. Seeds are treated with the mutagenic chemical and immediately planted to grow and produce selfed seed. Selfed generation after initial mutation are designated M1, M2 consecutively. Typically, the M2 seed will carry numerous new variations; therefore, no two experiments will produce the same combinations. Among these variations new traits previously not existing in rice and previously unavailable for selection by a plant breeder may be found and used for rice improvement.

To find new traits the breeder must use efficient and strategic selection strategies along the M generations, because the process is completely random and has an extremely low frequency of useful new combinations. Among thousands of induced new genetic variants, there may be only one with a desirable new trait. An optimal selection system will screen through thousands of new variants and allow detection of a few or even a single plant that might carry a new trait. After identifying or finding a possible new trait, the breeder must develop a new cultivar by pedigree or backcross breeding and extensive testing to verify the new trait and cultivar exhibits stable and heritable value to rice producers.

Targeted mutagenesis approaches, such as gene editing with clustered regularly interspaced short palindromic repeats (CRISPR), transcription activator-like effector nucleases (TALENs), meganucleases, or other types of endonucleases, represent a group of newer technique that can target specific genome regions for mutagenesis, leading to small insertions, deletion and even specific targeted sequence changes, that have much higher predictability of outcome. These techniques are now available to breeders for creating novel sequence modification in their target crop resulting in the desired phenotype. Collectively, targeted mutagenesis techniques, and also gene of interest insertion techniques via recombinant DNA techniques, represent key novel approaches to developing new climate resilient, highly productive varieties and hybrids, to meet global food demand.

### Herbicide Resistance in Rice

Weeds in crop fields compete for resources and greatly reduce the yield and quality of the targeted crop. Rice production is plagued by broad leaf as well as monocot weed problems during cultivation across growing areas. A particularly hard to control weed is called red rice. One difficulty arises because red rice is so genetically similar to cultivated rice (they occasionally cross pollinate) that there are no selective herbicides available that target red rice, yet do not harm the cultivated rice.

Weeds have been controlled in crops through the application of selective herbicides that kill the weeds, but do not harm the crop. Usually, selectivity of the herbicides is based on biochemical variations or differences between the crop and the weeds. The herbicides that are selective in nature, exert phytotoxicity only on certain specific group of plants, based on a specific susceptible interaction between their metabolism and the herbicide mode of action. Other herbicides, generally termed non-selective, exert phytotoxicity on every plant species/group, because they interfere with ubiquitous basic metabolisms shared by all plants and are used broadly globally across crops.

Dose effect in herbicide tolerance are not linearly related. Rice is naturally tolerant to low doses of isoxazolidinone class of pigment inhibitors such as 1-deoxy-D-xylulose 5-phosphate (DOXP) synthase inhibitors, diterpene inhibitors, and carotene inhibitors. In some embodiments, aforementioned inhibitors may include herbicides selected from Clomazone herbicide, bixlozone, or combination thereof. For that reason, diterpene inhibitors are already frequently used herbicides in this crop, as preemergent at low field doses, where they provide only moderate residuality and grass control. For this reason, its use must be complemented by application of other active ingredients to provide an effective weed control program for the crop.

With the widespread use of herbicides, high selection pressure is being applied in naturally occurring weed population favoring the selection of spontaneous mutant variants that can survive the herbicide application. Their reproductive success under selection leads to the emergence of herbicide resistant biotypes, that have emerged frequently, in multiple weed species, and multiple regions/instances, and are resistant to multiple different herbicide families and active ingredients. When weeds become resistant to a particular herbicide, that herbicide is no longer useful for weed control. This has led to targeted strategies to mitigate the emergence of herbicide resistant weed biotypes, as well as to advance the discovery of novel herbicide modes of action.

In turn, the understanding of the mutation and metabolic function of spontaneous weed resistance mechanism has resulted in the development of crop herbicide tolerance strategies by inserting, or modifying, the crop genome with genes or genetic variants that make the host plants tolerate the action of the herbicide. Resistance to herbicides was achieved in crops through recombinant gene technologies, and through induction of genetic mutations in-vivo that alter proteins and biochemical processes.

Herbicide tolerance mechanisms, in turn, are of two types, Target Site Resistance (TSR) and Non-Target Site Resistance (NTSR). TSR, refers to herbicide tolerance/tolerant (HT) mechanism in which the host plant has developed forms of the enzyme targeted by the herbicide mode of action, that render it immune to its actions, as compared to the native enzyme or protein. Through this TSR modifications the plants that were susceptible to the action of the herbicide become resistant to it. NTSR form of herbicide tolerance, on the other hand, do not involve the target site enzyme, but rather, involve new metabolic mechanisms that detoxify, compartmentalize, neutralize or otherwise limit the normal action of the herbicide preventing it from reaching the target site where it can exert a phytotoxic effect.

Weed control in commercial rice production is available already and was developed through random mutagenesis approaches, targeting mutations formerly described in naturally occurring weed populations. Finding new mutations in rice that confer resistant to other important herbicides is critical to enhance the tool available for effective weed management in rice. Similarly, discovery of new mutations that expand the tolerance level of the rice crop to herbicides for which it is naturally only marginally tolerant, is also highly desirable because this may eliminate the need to conduct additional herbicide applications, resulting in significant savings and simplification of the weed management programs. This document describes methods and systems that address some or all of the problems described above.

### SUMMARY

In one aspect, the disclosed technology relates to a monocot plant comprising a first mutation at chromosome 2 position 16835660 and/or a second mutation at chromosome 2 position 17087862, wherein the first mutation comprises a G to T single nucleotide mutation in cDNA position 595 resulting in amino acid change from aspartic acid to tyrosine at expressed protein position 199, wherein the second mutation comprises a G to T single nucleotide mutation in cDNA position 903 resulting in amino acid change from glutamine to histidine at expressed protein position 301, and wherein the monocot plant is tolerant/resistant to diterpene inhibitors, at levels higher than those tolerated by plants without either of the mutations. In some embodiments, the monocot plant is a rice plant. In some embodiments, the diterpene inhibitors is an isoxazolidinone class of diterpene inhibitors. In some embodiments, the diterpene inhibitor is selected from the group consisting of clomazone, bixlozone, and combinations thereof.

In some embodiments, seeds of the rice plant of present disclosure is deposited as ATCC accession number PTA-127860.

In some embodiments, the rice plant further comprises one or more genetic regions that confers tolerance/resistance to imidazolinone class of acetohydroxyacid synthase inhibiting herbicides, aryloxyphenoxypropionate class of acetyl-CoA carboxylase inhibitor herbicides, or triketone class of HPPD inhibiting herbicides.

In one aspect, the disclosed technology relates to a method for controlling weeds in a rice field, the method comprising: having rice in the field wherein the rice is tolerant/resistant to one or more diterpene inhibiting herbicide; and contacting the rice field with at least one of the diterpene inhibiting herbicide to which the rice is resistant at levels known to induce visible symptoms of phytotoxicity in rice plants, wherein the tolerance/resistance is associated with a mutation in genome of the rice at chromosome 2 position 16835660 comprising a G by T single nucleotide mutation in cDNA position 595 resulting in amino acid change from aspartic acid to tyrosine at expressed protein position 199, and/or a mutation at chromosome 2 position 17087862 comprising a G by T single nucleotide mutation in cDNA position 903 resulting in amino acid change from glutamine to histidine at expressed protein position 301, wherein herbicide rates greater than 34 oz/acre are known to induce visible symptoms of phytotoxicity, and wherein the visible symptoms of phytotoxicity comprise bleaching of leaf tissue, reduced growth rate, reduced plant height, delayed emergence, plant injury, or plant death.

In some embodiments, the method further comprises contacting the rice field with one or more additional herbicide selected from the group consisting of imidazolinone class of acetohydroxyacid synthase inhibiting herbicides, aryloxyphenoxypropionate class of acetyl-CoA carboxylase inhibiting herbicides, triketone class of HPPD inhibiting herbicides, and a combination thereof, wherein the one or more additional herbicide is premixed with the diterpene inhibiting herbicide before applying to the rice field or, wherein the additional herbicide is applied sequentially with the diterpene inhibiting herbicide before applying to the rice field.

In one aspect, the disclosed technology relates to a method of producing rice resistant to diterpene inhibitors, at levels significantly higher than those tolerated by plants without genetic regions conferring tolerance/resistance, the method comprising: obtaining genetic regions that are the equivalents of regions in genome of rice designated that confer tolerance/resistance; and introducing the genetic regions into rice lines to replace wild type regions, wherein the tolerance/resistance is associated with a mutation at chromosome 2 position 16835660 comprising a G by T single nucleotide mutation in cDNA position 595 resulting in amino acid change from aspartic acid to tyrosine at expressed protein position 199, and/or a mutation at chromosome 2 position 17087862 comprising a G by T single nucleotide mutation in cDNA position 903 resulting in amino acid change from glutamine to histidine at expressed protein position 301.

In some embodiments, the method comprises crossing the rice plant of present disclosure with a rice plant of a different genetic background and harvesting resultant hybrid rice seed.

In another aspect, the disclosed technology relates to an herbicide resistant rice plant produced from a rice seed produced by the method of the present disclosure. In another aspect, the disclosed technology relates to a rice plant, or a part thereof, produced by growing the seed of the present invention. In another aspect, the disclosed technology relates to a pollen or an ovule of the plant of the present disclosure.

In another aspect, the disclosed technology relates to a tissue culture produced from protoplasts or cells from the rice plant of the present disclosure, wherein said cells or protoplasts of the tissue culture are produced from a plant part selected from the group consisting of leaves, pollen, embryos, cotyledon, hypocotyl, meristematic cells, roots, root tips, pistils, anthers, flowers, stems, glumes and panicles. In another aspect, the disclosed technology relates to a protoplast produced from a plant of the present disclosure.

In another aspect, the disclosed technology relates to a method of producing a herbicide resistant rice plant; the method comprising modifying genome of a rice plant, wherein the modifying comprises introducing a mutation at chromosome 2 position 16835660 comprising a G by T single nucleotide mutation in cDNA position 595 resulting in amino acid change from aspartic acid to tyrosine at expressed protein position 199, and/or a mutation at chromosome 2 position 17087862 comprising a G by T single nucleotide mutation in cDNA position 903 resulting in amino acid change from glutamine to histidine at expressed protein position 301, and wherein the modifying confers resistance to an herbicide selected from the group consisting of clomazone, bixlozone, or a combination thereof. In some embodiments, the method comprises modifying the genome of the rice plant through use of gene editing proteins including meganucleases, TALENs, or CRISPR.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 depicts a scattergram showing results of a mutation mapping experiment (SNP index) for clomazone tolerant mutation on chromosome 2.

FIG. 2. shows a graphical representation of % crop injury 1 week post emergence of 3 high rate clomazone tolerant lines and a wild-type parent line (P1003) with no tolerance mutation.

FIG. 3 shows a graphical representation of % injury observed in experimental plots 3 weeks post pre-emergent herbicide application of Clomazone (wildtype control lines are indicated with “W”).

FIG. 4 shows a graphical representation of % bleaching observed in various lines 1 week post crop emergence following pre-emergent herbicide application of Clomazone.

FIG. 5 shows another graphical representation of % bleaching observed in various lines 1 week post crop emergence following pre-emergent herbicide application of Clomazone.

### DETAILED DESCRIPTION

Described and disclosed herein are novel and distinctive rice lines with unique resistances to herbicides, in particular, Diterpene Synthase inhibiting herbicides. Diterpene synthesis inhibiting herbicides include clomazone. For information on Clomazone and other herbicides, see “DEFINITIONS”

Rice lines having different herbicide resistance genes, either pyramided or stacked in the same genetic background or, as single products that are used alternatively in the rotation used by the farmer, represent a critical tool or strategy in extending the useful life of herbicides because these practices slow the development of herbicide resistant variants among the targeted weeds. Several methods are possible to deploy these resistances into hybrids or varieties for weed control, as well as options for hybrid seed production. The rice lines described herein represent new methods for weed control in rice and can be deployed in any of many possible strategies to control weeds and provide for long term use of these and other weed control methods.

Embodiments of the disclosed mutant rice lines are designated ML0831266-02958 and filed under ATCC deposit PTA-127860, with deposit application submitted on Dec. 13, 2024, henceforth referred to as “RTO1” rice line, which carry the mutations and are resistant/tolerant to high dose of preemergent diterpene inhibitors applications such as clomazone.

A putative set of mutations associated with resistance to diterpene inhibitors have been identified in rice chromosome 2, designated RTO1, indicating a putative NTSR mutation/s resulting in the enhanced tolerance to high dose herbicide. These mutations have not been reported before in rice. No report was found of any another rice mutation conferring resistance to high-dose clomazone applications. Also, there is no identified TSR mutation mechanism yet reported for diterpene synthesis inhibiting herbicides in rice nor in any other plant species.

An internally mutagenized population was developed and screened, at RiceTec, for diterpene inhibiting herbicide tolerance, allowing the identification of the mutagenized region of chromosome 2 that putatively confers tolerance to the herbicide used. The derived mutant tolerant lines were advanced and evaluated after the initial screening, to further characterize these new variants and assess their tolerance effect; confirm the mutation association with the novel phenotype and assess their commercial potential. Completion of these studies required several generations of population advancement and experimentation. These studies included multiple herbicide screening cycles, mutation mapping studies and DNA Marker linkage/analysis.

Compared with TSR, in which the herbicide resistance mechanism is directly associated to variation in the known target protein/gene whereby the metabolism of resistance is clearly inferred, NTSR mechanisms are much more difficult to characterize, and require extensive gene, gene action, and gene interaction studies. It is postulated that NTSR for this herbicide and others, could arise from onset of mechanisms triggering metabolic resistance such as compartmentalization, blockage of movement, detoxification, blockage of activation or other forms of preventing phytotoxicity. Also, recent studies showed that microRNAs (miRNAs) can be involved in the ‘gene regulation’ mechanisms leading to NTSR. In general, NTSR mechanisms and the molecular interaction involving the herbicide are more complex mechanisms than found in TSR.

A method to control weeds in a rice field, wherein the rice in the field includes plants resistant to a plurality of herbicides, and contacting the rice field with at least one herbicide, or a plurality of herbicides, for example, any that may belong to the family diterpene synthesis inhibitors, and/or any that may belong to the other classes of herbicides. In some embodiments, the other classes of herbicides may include acetohydroxyacid synthase (AHAS or ALS) inhibiting herbicides, acetyl-CoA carboxylase (ACCase) inhibiting herbicides, including broad-spectrum herbicides such as imidazolinone, aryloxyphenoxypropionates (FOPs), and triketone class of 4-hydroxyphenylpyruvate dioxygenase (HPPD) inhibiting herbicides, providing controls for a specific spectrum of weeds.

In some embodiments, diterpene inhibiting herbicide comprises isoxazolidinone class of herbicides including clomazone, bixlozone, or a combination thereof. In some embodiments, AHAS-inhibiting herbicides may comprise imidazolinone class of herbicides, ACCase-inhibiting herbicides may comprise aryloxyphenoxypropionate class of herbicides, and HPPD-inhibiting herbicides may comprise triketone class of herbicides. Further details regarding these herbicides and rice resistant to these herbicides are illustrated and described, for example, in U.S. Pat. No. 11,130,959 to Bernacchi et al., U.S. Pat. No. 11,877,553 to Bernacchi et al., and U.S. Pat. No. 9,303,270 to Hinga et al., the entire contents of which are incorporated herein by reference. A method for growing herbicide resistant/tolerant rice plants may include (a) planting resistant rice seeds; (b) allowing the rice seeds to sprout; (c) applying one or more herbicides to the rice sprouts at levels of herbicide that would normally inhibit the growth of a rice plant. In some embodiments, one or more additional herbicide is premixed with the diterpene inhibiting herbicide before applying to the rice field or the additional herbicide is applied sequentially with the diterpene inhibiting herbicide before applying to the rice field.

Methods of producing herbicide-tolerant rice plants may compromise obtaining genetic regions that are the equivalents of the regions in the genome of rice designated that confer tolerance/resistance; and introducing the genetic regions into rice lines to replace wild type regions. In some embodiments, the method may use a transgene or plurality of transgenes. One embodiment of such a method is transforming a cell of a rice plant with transgenes, wherein the transgenes encode 1, 2 or more different mutations each leading to resistance to diterpene inhibiting herbicides, e.g., clomazone, or other herbicides that confers tolerance in resulting rice plants to one or more herbicides. Any suitable cell may be used in the practice of these methods, for example, the cell may be in the form of a callus. In some embodiments, method of producing herbicide-tolerant rice may comprise transforming wild-type rice with genetic regions from any association of promotors, terminators, selectable markers.

In some embodiments, the method may also use an directed mutagenesis approach targeting single or a plurality of mutations. One embodiment of such a method is modifying a plant cell of a rice plant with directed mutagenesis methods, wherein the modified cell encodes 1, 2 or more different mutations each leading to resistance to diterpene inhibiting herbicides and/or other herbicides that confers tolerance in resulting rice plants to one or more herbicides. Any suitable cell may be used in the practice of these methods, for example, the cell may be in the form of a callus. Targeted mutagenesis approaches, such as gene editing with clustered regularly interspaced short palindromic repeats (CRISPR), transcription activator-like effector nucleases (TALENs), meganucleases, of other types of endonucleases represent a group of newer technique that can targets specific genome regions for mutagenesis, leading to small insertions, deletion and even specific targeted sequence changes, that with much higher predictability of outcome, are now available to breeder for creating novel sequence modification in their target crop resulting the desired herbicide resistant phenotype.

The mechanisms of herbicide tolerance in plants have been classified roughly into two groups: target-site and non-target-site herbicide tolerance. Target-site herbicide tolerance is generally caused by the prevention of herbicide binding to the target enzyme, caused by point mutations occurring in the target. On the other hand, non-target site herbicide tolerance targets other genes and metabolic processes that lead to a reduction of the lethality of the herbicides application dose, generally preventing it from reaching the target enzyme in its active form. The specific metabolic mechanisms may include inhibition of herbicide activation, herbicide detoxification, decrease of herbicide penetration, and herbicide sequestration or compartmentation in plant cells. Both TSR and NTSR herbicide tolerance factors have been used in agriculture in multiple crops and have been key factors in increasing agricultural output. Identification is novel NTSR mechanisms is desirable for as a complementation to existing TSR or when TSR option may not be available.

Rice lines having different herbicide resistance genes, either pyramided or stacked in the same genetic background or, as single products that are used alternatively in the rotation utilized by the farmer, represent a critical tool or strategy in extending the useful life of herbicides because these practices slow the development of herbicide resistant variants among the targeted weeds. Several methods are possible to deploy these resistances into hybrids or varieties for weed control, as well as options for hybrid seed production. The rice lines described herein represent new methods for weed control in rice, high dose clomazone tolerance, and can be deployed in any of many possible strategies to control weeds individually or in combination with other herbicide tolerance variants and provide for long-term use of these and other weed control methods. In particular, mutant rice tolerant to clomazone is disclosed.

Through developing sources of resistance to a high dose of clomazone the rice lines claimed provide the ability to extend the use of this herbicide for enhanced weed spectrum control, enhanced timing of controls, higher weed lethality supporting more effective weed resistance management, and potentially reducing the total number of applications and use of other active ingredients. The use of these rice lines including combining lines with resistance to herbicide with other modes of causative action, provides new options for weed control in grower's fields thus slowing the development of herbicide resistant weeds. Several methods are possible to deploy this resistance in hybrids for weed control as well as options for hybrid seed production, with different mutations.

Cells derived from herbicide resistant seeds, plants grown from such seeds and cells derived from such plants, progeny of plants grown from such seed and cells derived from such progeny are within the scope of this disclosure. The growth of plants produced from deposited seeds, and progeny of such plants will typically be resistant/tolerant to herbicides, e.g. clomazone, at levels of herbicides that would normally inhibit the growth of a corresponding wild-type rice plant. There are some natural (non-induced) levels of tolerance to some herbicides, but not protecting plants at levels that would be commercially useful.

A method for controlling growth of weeds in the vicinity of herbicide resistant/tolerant rice plants is also within the scope of the disclosure. One example of such methods is applying one or more herbicides to the fields of rice plants at levels of herbicide that would normally inhibit the growth of a rice plant. For example, at least one herbicide inhibits Diterpene synthesis activity. In order to maximize weed control in a rice field, different herbicides may be required to cover the spectrum of weeds present and, in turn, several applications along the crop cycle may be required for any one particular herbicide depending on the overlap between the window of effective control provided by a single application and the window of time during which its target weed may germinate, which often is longer than the protection afforded by a single herbicide application. Temperature, soil composition, and soil moisture conditions are key factors that affect both the window of herbicide efficacy and the moment of weed seed germination and growth. Based on these factors, herbicide control models often include sequential repeated application during the crop cycle.

Rice production for good yields requires specific weed control practices. Some herbicides are applied as preemergent, after planting but before crop emergence; other as postemergents. In the case of rice, postemergent application can be before the crops are flooded or after. Preferred applications are normally timed, according to the developmental stage of the crop, as defined by the number of open leaves in the growing plant (Table 1: Developmental Stages of Rice). Timing of herbicide applications is an important factor, not only from the perspective of maximizing the efficiency of weed control, but also from the perspective of minimizing impact on the herbicide tolerant crop. This consideration stems from the fact that mutagenized, naturally occurring or transgenic herbicide resistances often are not completely independent of dose and application timing effects. Different genes of herbicide resistance have different dose responses, as well as timing of application responses whereby, typically, phytotoxicity in the resistant crop increases as dose increases beyond a certain level, or phytotoxicity to the resistant crop varies with varying timings of application for a given herbicide.

Evaluation of the novel herbicide resistance genes, subject of this application, was conducted with a range of suitable herbicide doses, that cover application rates typically used for rice farming operations while also taking into consideration possible deviations from the manufacturer-recommended doses. Considering 1×, the recommended manufacturers or best practice recommended dose, the most frequently evaluated additional doses are 2× and 8× with some experiments including other values. A reference to dose by products and active ingredient content is provided in Table 2.

### Mutation Population Establishment and Screening

An Indica-type male line P1003, showing adaptation to US growing conditions, was incorporated into the RiceTec Breeding Program and used in population development, showing good combining ability and was chosen to undergo mutagenesis for discovery of herbicide tolerance traits.

A mutation breeding program was initiated in 2008, to develop proprietary herbicide resistant/tolerant lines using this line. A permanent mutant population was created by exposing approximately 500,000 seeds (estimated by the average weight of a kernel) of the line to mutagen sodium azide (AZ) and MNU. The treated seeds were planted after treatment. Individual plants were harvested creating approximately 3500 potentially mutation lines. The lines have been maintained by selfing as a permanent mutant population for trait screening.

The mutagenesis population were grown during 2021, under field condition with application of preemergent clomazone herbicide applied immediately after planting at a dose of 2520 gr ai/ac. See Example 1.

Plant material was evaluated for no-germination or injury 21 data after emergence of the untreated control. Surviving material was harvested for candidate line advancement.

Table 3 lists criteria for categorizing % injury in the plant sprayed (contacted) with a herbicide.

### Validation of Enhanced Tolerance/Resistance to Clomazone

After screening a large mutant population, several lines survived application of the herbicide. The surviving lines were increased to obtain sufficient seed for larger trials to further evaluate their tolerance to this herbicide. The tolerance in the line designated as ML0831266-02958, was validated by planting them in field plots (5 feet by 10 feet) with the non-mutant parent line P1003, and a second non-mutant line ML0831266-02958 as controls. The clomazone herbicide was applied as preemergent and a rate of 2520 grams (88.90 oz) of active ingredient per acre. 21 days after emergence, the plots were evaluated for percent injury caused to the rice based on control plots that had no herbicide application. The data confirms the tolerance of lines ML0831266-02958. In two subsequent experiment cycles, conducted during 2022 and 2023 similar experiments were conducted, exposing the selected tolerant lines progeny to the same clomazone preemergent application, to advance line purification and reconfirm their response.

### Identification of Putative Causal Mutations

The mutation mapping strategy was employed to identify the chromosomal location of the putative causal mutation causing tolerance to high dose applications of clomazone. Mutation mapping requires that a mutation mapping population be developed with a non-mutated parent, the F1 was selfed, and the derived F2 population be subject to herbicide selection and genomic sequencing. For this purpose, the mutant line ML0831266-02958 was crossed back to the original non-mutant parent P1003; their F1 progeny of the cross were selfed to produce a F2 population that is segregating for the tolerance causing mutation. Only mutations are segregating in this population because the mutations are the only genomic difference between ML0831266-02958 and wild-type P1003.

The F2 population was planted as individuals, and leaf tissue was collected with DNA extracted from each individual to use for genotyping after the population was phenotyped. The clomazone herbicide was applied to the F2 population as preemergent and at a concentration of 2520 gr ai/ha. Individuals that survived the herbicide application were scored as tolerant and those that died were scored as susceptible.

The DNA derived from a set of 30 surviving F2 individuals and 30 that were killed was each respectively bulked together and sequenced along with both the mutant line ML0831266-02958 and the non-mutant parent line P1003. Mapping the causal mutation was based on an index accessing the frequency of all mutations in the bulk representing the surviving individuals. The index was derived from the proportion of sequencing reads that carried a variation different from the non-mutant parent line. The more sequencing reads with the variation the closer the index was to one and if all sequencing reads had the variation the index equaled one.

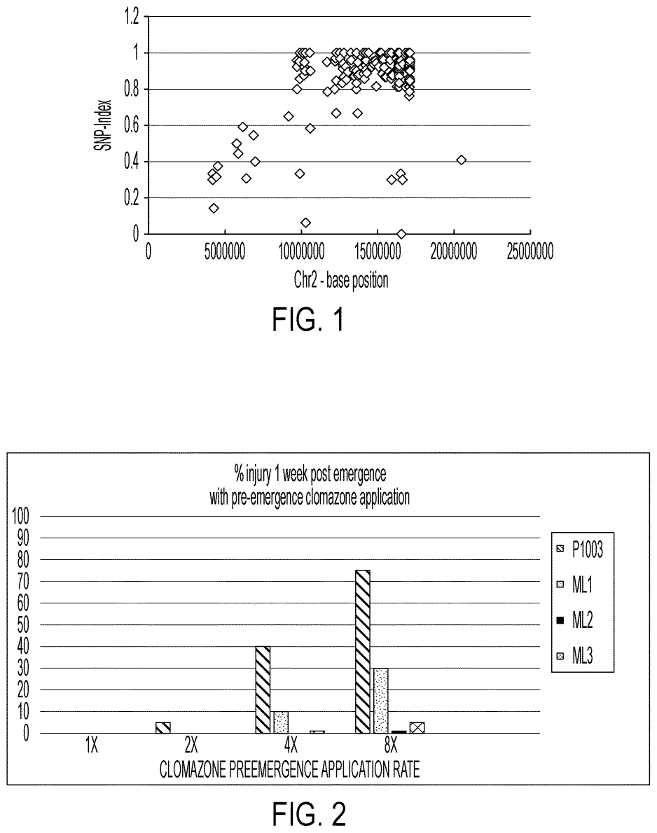

The analysis of these results showed one group including mutations on chromosome 2 with high single nucleotide polymorphism (SNP) Index scores only in the herbicide resistant bulk as compared to the wild type, indicative of phenotype association with the single plant bulking criteria utilized for sequencing, i.e. the Tolerant and Susceptible reaction the clomazone, (FIG. 1). Excluding intronic, intergenic, and synonymous mutations, only 12 mutations were detected, within this chromosome 2 group, that are non-synonyms and expressed, (Table 4). Of these 12 mutations, 7 mutations correspond to transposon elements (Positions 9700994; 14214751; 14233942; 14419550; 15340535; 15382925 and 15814105); and 2 other mutations (Positions 15362450 and 16390954) are of unknown type and have lower SNP index, and another mutation (Position 16404758) is and NB-ARC domain containing protein involved in plant disease resistance response.

The remaining 2 mutations detected, had SNP Index of 1, at positions 16835660 and 17087862 corresponding respectively to non-synonymous mutations and are therefore considered the primary candidates for putative causal effect of the high dose clomazone tolerance. The mutation at chromosome 2 position 16835660, is in gene LOC_Os02g28470, in exon1, showing a G by T single nucleotide mutation in cDNA position 595 resulting in amino acid change of aspartic acid to tyrosine at the expressed protein position 199 (D199Y). The mutation at chromosome 2 position 17087862 is in gene LOC_Os02g28870, in exon3, showing a G by T nucleotide change in cDNA position 903 corresponding to a glutamine (Q) to histidine (H) amino acid change in position 301 (G301H) of the expressed polypeptide.

### EXAMPLES

### Example 1: Initial Mutant Screening (21-T3)

A mutant population of inbred rice line P1003, representing 4754 individual mutant lines, was grown in the field and subjected to selection by pre-emergent application of the herbicide Clomazone, applied at a rate of 2520 g ai/ha (96 oz/ac of Vopak 3ME herbicide). 21 days after the herbicide application plants were assessed for phytotoxicity in response to the clomazone. Symptoms of high rate clomazone application are bleaching (white leaves and shoots) of leaves and reduced or delayed emergence. Low rates of emergence were observed across the field with extensive bleaching in most mutant lines. 21 individual plants exhibiting no bleaching with emergence and growth in line with control plots that were not subjected to herbicide application. These plants were categorized as tolerant to increased rates of clomazone herbicide, greater than current maximum tolerable rates for rice, and were selected for advancement.

### Example 2: Advancing Tolerant Lines (21GH-T5 & 22-T24)

Tolerant mutant lines harvested from the initial field screen were grown in a greenhouse and further subjected to a pre-emergent application of clomazone at a rate of 2520 g ai/ha (96 oz/ac of Vopak 3ME herbicide). Plants were assessed for rate of emergence and bleaching starting 1 week post application and continuing for 3 weeks. Plants exhibiting normal emergence and growth as compared to untreated controls along with no visible bleaching were identified as tolerant to increased rates of clomazone and selected for advancement. These tolerant lines were then planted in short rows in a field in Alvin, Texas and further subjected to a pre-emergent application of clomazone at a rate of 2520 g ai/ha (96 oz/ac of Vopak 3ME herbicide). During field observation lines were selected for vigorous growth with mutant lines most closely matching the phenotype of the non-mutated P1003 advanced.

### Example 3: Validation RTO1 Mutants 23-T17 Generation of Mapping Populations

Seed of increased rate clomazone tolerant lines was increased in Puerto Rico, under selective pressure of a pre-emergent application of clomazone at a rate of 2520 g ai/ha (96 oz/ac of Vopak 3ME herbicide) followed by field testing in Alvin, Texas. Negative selection of a pre-emergent application of clomazone at a rate of 2520 g ai/ha (96 oz/ac of Vopak 3ME herbicide) was used to identify a subset of lines for use in the generation of a genetic mapping population (FIG. 2). Multiple rates of clomazone herbicide were applied to small field plots at rates ranging from 315 g ai/ha (1×; 12 oz/ac Vopak 3ME herbicide) to 2520 g ai/ha (8×; 96 oz/ac of Vopak 3ME herbicide). A single application of the herbicide was made as a pre-emergent application.

### Example 4: Mapping RTO1 Mutation 24-T20

Mutagenesis was conducted in a rice line with sodium azide (AZ) & N-methyl-N-nitrosourea (MNU). The mutant population generated, in this case a semi dwarf inbred line, was screened with the herbicide and resistant surviving lines were selected over several cycles. These lines were crossed to a wild-type plant of the same cultivar and the resulting F1 was self-pollinated to obtain F2 progeny segregating for the mutant and wild-type phenotypes. Crossing of the mutant to the wild-type parental line ensured detection of phenotypic differences at the F2 generation between the mutant and wild type. DNA of the plants F2 displaying the mutant phenotype were bulked and subjected to whole genome sequencing, followed by alignment to the reference sequence. SNPs with sequence reads composed only of mutant sequences (SNP index of 1) are putatively closely linked to the causal SNP for the mutant phenotype.

The strongest indication of putative mutations associated with Clomazone resistance was observed by Chromosome 2, with a string of sequential mutations with the highest SNP Index, [FIG. 1, 10 key mutations in a row (SNP Index 1)]

### Example 5:24-T5 Dose Response of RTO1 Mutant Lines, Lines Selection

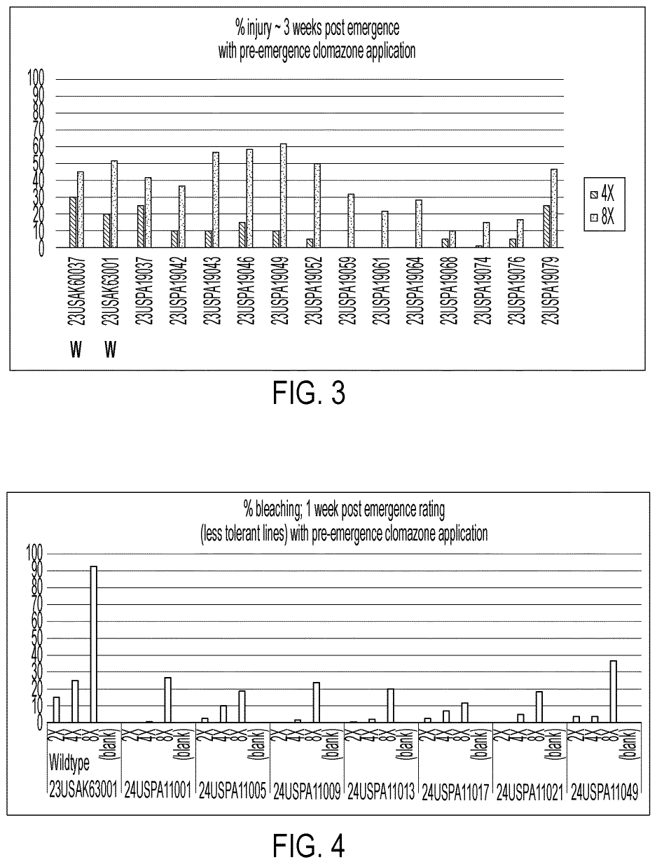

After large scale seed increase of tolerant mutant lines in Puerto Rico field testing in Alvin, Texas continued with planting of progeny lines derived from the original mutant lines selected. Plots received a pre-emergent application of clomazone herbicide at rates of 1260 g ai/ha (4×; 48 oz/ac of Vopak 3ME herbicide) or 2520 g ai/ha (8×; 96 oz/ac of Vopak 3ME herbicide). Mutant lines showed variable response to the clomazone herbicide (FIG. 3). One of the most tolerant lines, designated ML0831266-02958 (Source #23USPA19061), was selected as the trait donor for introgression of the increased rate clomazone tolerance trait designated RTO1. The mutant lines tolerant to increased rates of clomazone herbicide showed the same phenotype observed in initial screens with normal rates of emergence and growth rates, as compared to untreated control line P1003, and no bleaching of leaves at or post emergence. In contrast P1003 control plots had delayed emergence and reduced growth rate. Significant leaf bleaching is still visible 4 weeks post pre-emergent application.

### Example 6: Production of Hybrid Rice Resistant to Increased Rates of Diterpene Synthesis Inhibitor Herbicides and Elimination of Non-Tolerant Selfed Seed

The Diterpene synthesis inhibitor herbicide tolerance designated RTO1 and provided by ML0831266-02958 is deployed individually into hybrids through either the male or female parent resulting in the hybrid seed being resistant to the herbicide. If the resistance is deployed in only the male parent, then in addition to its use for weed control, the herbicide when applied to hybrid seed kills contaminating female selfed seed. On the other hand, if the resistance is deployed only through the female parent, growers may eliminate contaminating male selfed seed.

### Example 7: Production of Hybrid Rice Resistant to Increased Rates of Diterpene Synthesis Inhibitor Herbicides

The Diterpene synthesis inhibitor herbicide tolerance designated RTO1 and provided by ML0831266-02958 is deployed into the male and/or female parent of a hybrid. The resulting hybrid seed may carry resistance to clomazone or other Diterpene synthesis inhibitor herbicides.

### Example 8: Production of Hybrid Rice Resistant to Increased Rates of Diterpene Synthesis Inhibitor Herbicides and at Least One Other Herbicide Class

Resistance to Diterpene synthesis inhibitor herbicides and at least one other herbicide class is deployed in a single hybrid by using a male parent that carries resistance to clomazone (or other herbicide) and a female that carries the other resistance. The method allows the grower to make a single purchase but to be able to choose which herbicide to apply. A single class of herbicide may be used in any one season and rotated between seasons, or alternatively both herbicides could be applied within a single season. In addition, deployment by this method eliminates contaminating selfed seed of both parents in the hybrid seed through application of both herbicides, or one type or the other, are eliminated through application of only one herbicide.

In another method of deployment, the clomazone resistance and any other herbicide resistance is deployed through the making a hybrid with a male parent that carries both resistances. The grower then has the option to choose which herbicide class to apply or to apply both within a single season. In addition, through the application of either herbicide contaminating selfed female seed would be eliminated. Alternatively, both herbicide class resistances are provided in the female parent, giving the grower the same options for weed control.

Another embodiment is to deploy the clomazone resistance to both parents, and another herbicide resistance into only one parent, such as the male parent. The hybrid seed are then homozygous for the clomazone resistance but not the other. A scheme like this is used to make an early application with the herbicide put into only the male parent, providing weed control and elimination of contaminating female selfs. Later in the season clomazone may be applied or another diterpene synthesis inhibitor herbicide. The useful life of both herbicides is extended through limiting or eliminating the development of weed resistance. In another application this method allows the use of clomazone at rates above current label maximums to control weeds in seed production fields, allowing for cleaner seed.

Alternatively, a different herbicide resistance could be deployed in both the male and female hybrid parent and the diterpene synthesis inhibitor resistance is deployed in only the male parent.

Other embodiments for deploying herbicide resistant lines include other traits such as resistance to other classes of herbicides, or other traits of importance.

### Example 9: Production of a Rice Variety Resistant to Increased Rates of Diterpene Synthesis Inhibitor Herbicides

The Diterpene synthesis inhibitor herbicide tolerance designated RTO1 and provided by ML0831266-02958 is deployed in a rice variety through the introduction of the RTO1 trait during a trait introgression process. Trait introgression may include the introduction of the RTO1 trait into rice varieties through crossing ML0831266-02958 or derivatives therefrom and using phenotypic and/or genotypic selections to identify progeny that has acquired the RTO1 trait. Additional backcrossing and selfing generations may be used to fix the trait and eliminate any undesirable phenotypic characteristics of the RTO1 donor line. Furthermore, the RTO1 trait may be introduced directly to rice varieties through methods other than sexual crossing including the introduction of a transgene encoding for RTO1 or the use of gene editing technology to directly introduce the RTO1 mutation in a rice variety.

### Example 10: Seed Productivity and High-Dose Response of Advancing Selection of Diterpene Synthesis Inhibitor Herbicides Tolerance

13 advanced self selections, derived from ML0831266-02958 were increased and tested during winter 2024 in Puerto Rico, and sprayed with 8× field dose of herbicide Clomazone. Some differences in crop response was noted between sources, but generally plants responded well to the Clomazone application with minimal bleaching. Plant growth was good throughout the season. At harvest all plots of the same planted source were harvested as bulks and combined. Seed production of all sources was commensurate with wild type material. (Table 5).

### Example 11: Dose-Response of Advance Selections of Rice with High-Dose Diterpene Synthesis Inhibitor Herbicides Tolerance

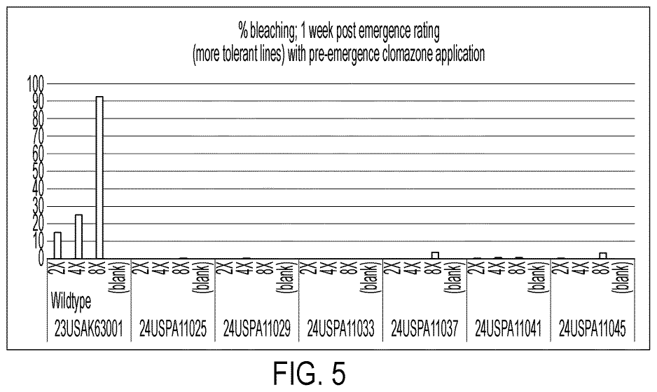

A total of 13 selections of from original high-dose clomazone mutant line ML0831266-02958 were advanced for further testing. These were grown in a replicated experiment in Alvin TX, 2025, and tested with preemergent application of Clomazone at 2×, 4× and 8× field dose, applied 1 day after planting. % bleaching of plants in plots was assessed for 3 consecutive weeks starting ˜1 week post emergence. Standard field practices were used to care for the plants. FIG. 4 and FIG. 5 show data for these 13 selections. FIG. 4 shows the reaction of seven selections with moderate bleaching between 10% and 40% at 8× field dose compared to the wild type control (line 23USAK63001) as measured 1 week post emergence. FIG. 5 shows the reaction of the six highest performing (most tolerant) line selections that showed significantly reduced levels of bleaching below 5%, as measured 1 week post emergence and which had fully recovered by the second week after applications, even at the 8× herbicide rate. These 6 line selections were all siblings from a specific prior advancement, and showed minimal plant to plant variation in response. Other selection showed higher bleaching and intra plot variability indicating residual segregation. Wild type control plots showed high rates of bleaching, at 8× rates, 1 week post emergence following pre-emergent application prior to onset of extensive plant death. Germination rates and timing in tolerant mutant lines were on par with wildtype control.

Based on these results, a strong, dominant allele has been identified that provides increased tolerance to Clomazone. As can be observed in FIG. 5 there are 6 lines that show very little bleaching even up to 8× rates. In these plots the bleaching was the result of few individual plants and was not widespread. Beyond the few individual plants there was no other symptomology observed in these plots and emergence was in line with unsprayed control plots. Initial plant growth was vigorous in these plots. Lines in FIG. 5 upon closer pedigree analysis proved to be sub-lines reinforcing the probability that the allele these lines contain is a true trait of value. Other lines that showed higher bleaching response represented different mutant pedigrees and would likely contain alternative mutations. In contrast to the positive mutants the sensitive control line 23USAK63001 (P1003) was stunted with nearly all plants exhibiting bleaching or chlorosis in response to the 8× rate.

### Seed Deposits Under Budapest Treaty

Representative sample of seed was deposited by RiceTec Inc. with the American Type Culture Collection (ATCC), 10801 University Boulevard, Manassas, Va 20110, United States of America. The date of deposit and the ATCC Accession Number is PTA-127860, with deposit application submitted on Dec. 13, 2024. All restrictions will be removed upon granting of a patent, and the deposits are intended to meet all of the requirements of 37 C.F.R. §§ 1.801-1.809, and satisfy the Budapest Treaty requirements. The deposit will be maintained in the depository for a period of thirty years, or five years after the last request, or for the enforceable life of the patent, whichever is longer, and will be replaced as necessary during that period.

### Definitions

In the description and tables which follow, a number of terms are used. In order to provide a clear and consistent understanding of the specification and claims, including the scope to be given such terms, the following definitions are provided:

Allele: Allele is any one of many alternative forms of a gene, all of which generally relate to one trait or characteristic. In a diploid cell or organism, the two alleles of a given gene occupy corresponding loci on a pair of homologous chromosomes.

Backcrossing: Process of crossing a hybrid progeny to one of the parents, for example, a first generation hybrid F1 with one of the parental genotypes of the F1 hybrid.

Blend: Physically mixing rice seeds of a rice hybrid with seeds of one, two, three, four or more of another rice hybrid, rice variety or rice inbred to produce a crop containing the characteristics of all of the rice seeds and plants in this blend.

Cell: Cell as used herein includes a plant cell, whether isolated, in tissue culture or incorporated in a plant or plant part.

Cultivar: Variety or strain persisting under cultivation.

Diterpene inhibitors: Deoxy-D-Xylulose phosphate synthase (DOXP) inhibitor and carotene inhibitors are used somewhat interchangeably herein.

Embryo: The embryo is the small plant contained within a mature seed.

Essentially all the physiological and morphological characteristics: A plant having essentially all the physiological and morphological characteristics of the hybrid or cultivar, except for the characteristics derived from the converted gene.

Grain Yield: Weight of grain harvested from a given area. Grain yield could also be determined indirectly by multiplying the number of panicles per area, by the number of grains per panicle, and by grain weight.

Injury to Plant: Is defined by comparing a test plant to controls and finding the test plant is not same height; an abnormal color, e.g. yellow not green; a usual leaf shape, curled, fewer tillers.

Locus: A locus is a position on a chromosome occupied by a DNA sequence; it confers one or more traits such as, for example, male sterility, herbicide tolerance, insect resistance, disease resistance, waxy starch, modified fatty acid metabolism, modified phytic acid metabolism, modified carbohydrate metabolism and modified protein metabolism. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Induced: As used herein, the term induced means genetic resistance appeared after treatment with mutagen.

Non-induced: As used herein, the term non-induced means genetic resistance not known to be induced; is at different location in the genome, than induced resistance.

Plant: As used herein, the term “plant” includes reference to an immature or mature whole plant, including a plant from which seed or grain or anthers have been removed. Seed or embryo that will produce the plant is also considered to be the plant.

Plant Part: As used herein, the term “plant part” (or a rice plant, or a part thereof) includes protoplasts, leaves, stems, roots, root tips, anthers, pistils, seed, grain, embryo, pollen, ovules, cotyledon, hypocotyl, glumes, panicles, flower, shoot, tissue, cells, meristematic cells and the like.

Quantitative Trait Loci (QTL): Genetic loci that controls to some degree numerically measurable traits that are usually continuously distributed.

Recombinant/Non-Recombinant: If non-parental combination occurs, a rice plant is recombinant.

Regeneration: Regeneration refers to the development of a plant from tissue culture.

Resistance/Resistant: The inherited ability of a plant to survive and reproduce following exposure to a dose of herbicide normally lethal to the wild type resistance may be naturally occurring or induced by such techniques as genetic engineering or selection of variants produced by tissue culture or mutagenesis.

Single Gene Converted (Conversion): Single gene converted (conversion) includes plants developed by a plant breeding technique called backcrossing wherein essentially all of the desired morphological and physiological characteristics of an inbred are recovered, while retaining a single gene transferred into the inbred via crossing and backcrossing. The term can also refer to the introduction of a single gene through genetic engineering techniques known in the art.

Stacking: Adding more than one thing to the same receiving entity. Methods of achieving the “stacked” state include methods of vector-stack two or more genes in a single vector and do a single transformation to achieve stack; do sequential transformations into same receptor adding traits stepwise; achieve stacked hybrid simply by end crossing parentals carrying different traits; develop lines with multiple traits by sequential mutagenesis or crossing, and fixing the stacked state into one parent; and variants thereof.

Tolerance/Tolerant: The inherent ability of a species to survive and reproduce after herbicide treatment implies that there was no selection or generic manipulation to make the plant tolerant. Resistance/Resistant are used somewhat interchangeably herein; for a specific rice plant genotype information is provided on the herbicide applied, the strength of the herbicide, and the response of the plant.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

Tables

TABLE 2
Reference Herbicide Products used, and Doses

| | | Active | Product | Use | | | | |
|---|---|---|---|---|---|---|---|---|
| Product | Active | Ingredient | Concentration | rate | 1X | 2X | 4X | 8X |
| Name | Ingredient | Concentration | Unit | units | Dose | Dose | Dose | Dose |
| Vopak ® | Clomazone | 35.9 | gm ai/100 ml | g ai/ha | 315 | 630 | 1260 | 2520 |
| 3ME | | | | | | | | |
| Vopak ® | Clomazone | 35.9 | gm ai/100 ml | oz/acre | 12 | 24 | 48 | 96 |
| 3ME | | | | | | | | |

TABLE 3
Herbicide Injury Rating Scale in Rice

| Score | Rating description |
|---|---|
| 0 | No visible injury |
| 1 | Injury observed in at least 1 plant but very minimal |
| 5 | Minimal injury observed across plot |
| 10 | Plants are stunted 10% as compared to control, or plants show |
| | herbicide injury on approximately 10% of leaf area in the plot |
| 15 | Plants are stunted 15% as compared to control, or plants show |
| | herbicide injury on approximately 15% of leaf area in the plot |
| 20 | Plants are stunted 20% as compared to control, or plants show |
| | herbicide injury on approximately 20% of leaf area in the plot |
| 25 | Plants are stunted 25% as compared to control, or plants show |
| | herbicide injury on approximately 25% of leaf area in the plot |
| 30 | Plants are stunted 30% as compared to control, or plants show |
| | herbicide injury on approximately 30% of leaf area in the plot |
| 35 | Plants are stunted 35% as compared to control, or plants show |
| | herbicide injury on approximately 35% of leaf area in the plot |
| 40 | Plants are stunted 40% as compared to control, or plants show |
| | herbicide injury on approximately 40% of leaf area in the plot |
| 45 | Plants are stunted 45% as compared to control, or plants show |
| | herbicide injury on approximately 45% of leaf area in the plot |
| 50 | Plants are stunted 50% as compared to control, or plants show |
| | herbicide injury on approximately50% of leaf area in the plot |
| 55 | Plants show herbicide injury on approximately 55% of leaf |
| | area in the plot |
| 60 | Plants show herbicide injury on approximately 60% of leaf |
| | area in the plot |
| 65 | Plants show herbicide injury on approximately 65% of leaf |
| | area in the plot |
| 70 | Plants show herbicide injury on approximately 70% of leaf |
| | area in the plot |
| 75 | Plants show herbicide injury on approximately 75% of leaf |
| | area in the plot |
| 80 | Plants show herbicide injury on approximately 80% of leaf |
| | area in the plot |
| 85 | Plants show herbicide injury on approximately 85% of leaf |
| | area in the plot |
| 90 | Plants show herbicide injury on approximately 90% of leaf |
| | area in the plot |
| 95 | All plants severely injured, most are dead. Some green tissue |
| | spread throughout plot. |
| 99 | Nearly all plants are dead, but at least 1 plant has green tissue. |
| 100 | All plants dead and brown. No green tissue in the plot. |

TABLE 4
Identified Mutations in Chromosome 2

| | Ref. | Alt. | | | | | Gene Function |
|---|---|---|---|---|---|---|---|
| Position | Allele | Allele | SNP_index | SNP_Function | Gene: AAChange | Remarks | annotation |
| 9700994 | C | T | 0.9583 | nonsynonymous | LOC_Os02g16990:LOC_{—} | Unknown | retrotransposon |
| | | | | SNV | Os02g16990.1:exon7:c.G1414A:p.A472T | | protein, |
| | | | | | | | putative, |
| | | | | | | | unclassified, |
| | | | | | | | expressed |
| 14214751 | G | A | 0.9630 | nonsynonymous | LOC_Os02g24530:LOC_{—} | Unknown | retrotransposon |
| | | | | SNV | Os02g24530.1:exon1:c.C701T:p.T234M | | protein, |
| | | | | | | | putative, |
| | | | | | | | unclassified, |
| | | | | | | | expressed |
| 14233942 | G | A | 0.9583 | nonsynonymous | LOC_Os02g24560:LOC_{—} | Unknown | retrotransposon |
| | | | | SNV | Os02g24560.1:exon4:c.G389A:p.R130H | | protein, |
| | | | | | | | putative, |
| | | | | | | | unclassified, |
| | | | | | | | expressed |
| 14419550 | G | A | 1.0000 | nonsynonymous | LOC_Os02g24870:LOC_{—} | Unknown | retrotransposon |
| | | | | SNV | Os02g24870.1:exon2:c.C205T:p.L69F | | protein, |
| | | | | | | | putative, |
| | | | | | | | Ty3-gypsy |
| | | | | | | | subclass |
| 15340535 | A | T | 0.9688 | nonsynonymous | LOC_Os02g26110:LOC_{—} | Unknown | retrotransposon |
| | | | | SNV | Os02g26110.1:exon3:c.T2987A:p.F996Y | | protein, |
| | | | | | | | putative, |
| | | | | | | | unclassified, |
| | | | | | | | expressed |
| 15362450 | G | A | 0.9524 | nonsynonymous | LOC_Os02g26150:LOC_{—} | Unknown | hypothetical |
| | | | | SNV | Os02g26150.1:exon1:c.G142A:p.G48R | | protein |
| 15382925 | C | T | 0.9615 | nonsynonymous | LOC_Os02g26190:LOC_{—} | Unknown | transposon |
| | | | | SNV | Os02g26190.1:exon12:c.C3794T:p.A1265V, | | protein, |
| | | | | | LOC_Os02g26190:LOC_{—} | | putative, |
| | | | | | Os02g26190.2:exon12:c.C3794T:p.A1265V | | CACTA, |
| | | | | | | | En/Spm sub- |
| | | | | | | | class, |
| | | | | | | | expressed |
| 15814105 | G | A | 0.9600 | nonsynonymous | LOC_Os02g26920:LOC_{—} | Unknown | transposon |
| | | | | SNV | Os02g26920.1:exon2:c.G3109A:p.E1037K | | protein, |
| | | | | | | | putative, |
| | | | | | | | CACTA, |
| | | | | | | | En/Spm sub- |
| | | | | | | | class, |
| | | | | | | | expressed |
| 16390954 | C | T | 0.9583 | nonsynonymous | LOC_Os02g27660:LOC_{—} | Unknown | expressed |
| | | | | SNV | Os02g27660.1:exon1:c.G91A:p.G31S | | protein |
| 16404758 | G | A | 0.9524 | nonsynonymous | LOC_Os02g27680:LOC_{—} | function | NB-ARC |
| | | | | SNV | Os02g27680.1:exon2:c.G109A:p.D37N | analysis of | domain |
| | | | | | | the NB- | containing |
| | | | | | | ARC | protein |
| | | | | | | domain of | |
| | | | | | | plant | |
| | | | | | | disease | |
| | | | | | | resistance | |
| | | | | | | proteins | |
| 16835660 | G | T | 1.0000 | nonsynonymous | LOC_Os02g28470:LOC_{—} | Not well | transferase |
| | | | | SNV | Os02g28470.1:exon1:c.G595T:p.D199Y | characterized | family |
| | | | | | | | protein, |
| | | | | | | | putative, |
| | | | | | | | expressed |
| 17087862 | G | T | 1.0000 | nonsynonymous | LOC_Os02g28870:LOC_{—} | The U-box- | PUB73, |
| | | | | SNV | Os02g28870.1:exon3:c.G903T:p.Q301H | type E3 | OsPUB73: |
| | | | | | | ligase | U-box |
| | | | | | | OsPUB73 | domain- |
| | | | | | | interacts | containing |
| | | | | | | with and | protein, |
| | | | | | | promotes | putative |
| | | | | | | OsVQ25 | PUB gene |
| | | | | | | degradation | |
| | | | | | | via the | |
| | | | | | | UPS to | |
| | | | | | | positively | |
| | | | | | | regulate | |
| | | | | | | SNS BSR. | |
| | | | | | | The | |
| | | | | | | degradation | |
| | | | | | | of OsVQ25 | |
| | | | | | | by OsPUB73 | |
| | | | | | | also helps | |
| | | | | | | maintain | |
| | | | | | | proper plant | |
| | | | | | | growth as | |
| | | | | | | OsVQ25 can | |
| | | | | | | suppress | |
| | | | | | | plant | |
| | | | | | | development | |
| | | | | | | by inhibiting | |
| | | | | | | the activity of | |
| | | | | | | OsWRKY53. | |

TABLE 5
Clean Seed yield derived from ML0831266-02958

| Source # | Seed weight (Kg) |
|---|---|
| 24USPA11001 | 7.606 |
| 24USPA11005 | 7.635 |
| 24USPA11009 | 5.925 |
| 24USPA11013 | 6.537 |
| 24USPA11017 | 6.723 |
| 24USPA11021 | 5.473 |
| 24USPA11025 | 8.211 |
| 24USPA11029 | 9.999 |
| 24USPA11033 | 7.973 |
| 24USPA11037 | 6.85 |
| 24USPA11041 | 6.503 |
| 24USPA11045 | 6.703 |
| 24USPA11049 | 5.213 |

### PUBLICATIONS

All publications cited in this application are herein incorporated by reference to the extent they relate materials and/or methods related to the invention.
Agarwal et al., Plant Mol Biol (2007) 65:467-485. Genome-wide identification of C2H2 zincfinger gene family in rice and their phylogeny and expression analysis.
Akira, Abe et al., Genome sequencing reveals agronomically important loci in rice using MutMap Nature Biotechnology 30,174-178 (2012), Published online 22 Jan. 2012.
Wright, Mark H. et al., Genome-wide association mapping reveals a rich genetic architecture of complex traits in Oryza sativa. Nat. Comm 2:467 | DOI: 10.1038/ncomms1467, Published Online 13 Sep. 2011. The marker information can be accessed from The Rice Diversity Home Page and downloading the file “44K GWAS Data” (http://www.ricediversity.org/index.cfm).
Zhao, Keyan et al. (2011). Genome-wide association mapping reveals a rich genetic architecture of complex traits in Oryza sativa. Nat Comm 2:467 | DOI: 10.1038/ncomms1467, Published Online 13 Sep. 2011.

What is claimed is:

1. A monocot plant comprising a first mutation at chromosome 2 position 16835660 and/or a second mutation at chromosome 2 position 17087862,
wherein the first mutation comprises a G to T single nucleotide mutation in cDNA position 595 resulting in amino acid change from aspartic acid to tyrosine at expressed protein position 199,
wherein the second mutation comprises a G to T single nucleotide mutation in cDNA position 903 resulting in amino acid change from glutamine to histidine at expressed protein position 301, and
wherein the monocot plant is tolerant/resistant to diterpene inhibitors, at levels higher than those tolerated by plants without either of the mutations.

2. The monocot plant of claim 1, wherein the monocot plant is a rice plant.

3. The monocot plant of claim 1 wherein the diterpene inhibitors is an isoxazolidinone class of diterpene inhibitors.

4. The monocot plant of claim 1 wherein the diterpene inhibitor is selected from the group consisting of clomazone, bixlozone, and combinations thereof.

5. Seeds of the rice plant of claim 2 deposited as ATCC accession number PTA-127860.

6. The monocot plant of claim 2, where the rice plant further comprises one or more genetic regions that confers tolerance/resistance to imidazolinone class of acetohydroxyacid synthase inhibiting herbicides, aryloxyphenoxypropionate class of acetyl-CoA carboxylase inhibitor herbicides, or triketone class of HPPD inhibiting herbicides.

7. A method for controlling weeds in a rice field, the method comprising:
a) having a rice plant in the rice field wherein the rice is tolerant/resistant to one or more diterpene inhibiting herbicide; and
b) contacting the rice field with at least one of the diterpene inhibiting herbicide to which the rice is resistant at levels known to induce visible symptoms of phytotoxicity in rice plants,
wherein the tolerance/resistance is associated with a mutation in genome of the rice at chromosome 2 position 16835660 comprising a G by T single nucleotide mutation in cDNA position 595 resulting in amino acid change from aspartic acid to tyrosine at expressed protein position 199, and/or a mutation at chromosome 2 position 17087862 comprising a G by T single nucleotide mutation in cDNA position 903 resulting in amino acid change from glutamine to histidine at expressed protein position 301, and
wherein the visible symptoms of phytotoxicity comprise bleaching of leaf tissue, reduced growth rate, reduced plant height, delayed emergence, plant injury, or plant death.

8. The method of claim 7, wherein the diterpene inhibiting herbicide comprises clomazone, bixlozone, or a combination thereof.

9. The method of claim 7, further comprising contacting the rice field with one or more additional herbicide selected from the group consisting of imidazolinone class of acetohydroxyacid synthase inhibiting herbicides, aryloxyphenoxypropionate class of acetyl-CoA carboxylase inhibiting herbicides, triketone class of HPPD inhibiting herbicides, and a combination thereof,
wherein the one or more additional herbicide is premixed with the diterpene inhibiting herbicide before applying to the rice field or,
wherein the additional herbicide is applied sequentially with the diterpene inhibiting herbicide before applying to the rice field.

10. A method of producing rice resistant to diterpene inhibitors, at levels significantly higher than those tolerated by plants without genetic regions conferring tolerance/resistance, the method comprising:
a) obtaining genetic regions that are equivalents of regions in genome of rice designated that confer tolerance/resistance; and
b) introducing the genetic regions into rice lines to replace wild type regions,
wherein the tolerance/resistance is associated with a mutation at chromosome 2 position 16835660 comprising a G by T single nucleotide mutation in cDNA position 595 resulting in amino acid change from aspartic acid to tyrosine at expressed protein position 199, and/or a mutation at chromosome 2 position 17087862 comprising a G by T single nucleotide mutation in cDNA position 903 resulting in amino acid change from glutamine to histidine at expressed protein position 301.

11. A method for producing a rice seed, wherein the method comprises crossing the rice plant of claim 2 with a rice plant of a different genetic background and harvesting resultant hybrid rice seed.

12. An herbicide resistant rice plant produced from a rice seed produced by the method of claim 11.

13. A rice seed wherein a representative sample of seed was deposited under ATCC Accession No. PTA-127860.

14. A rice plant, or a part thereof, produced by growing the seed of claim 13.

15. Pollen or an ovule of the plant of claim 14.

16. A rice plant, or a part thereof, having all of physiological and morphological characteristics of the rice plant of claim 12.

17. A tissue culture produced from protoplasts or cells from the rice plant of claim 12 wherein said cells or protoplasts of the tissue culture are produced from a plant part selected from the group consisting of leaves, pollen, embryos, cotyledon, hypocotyl, meristematic cells, roots, root tips, pistils, anthers, flowers, stems, glumes and panicles.

18. A protoplast produced from a plant of claim 14.

19. A method of producing a herbicide resistant rice plant; the method comprising modifying genome of a rice plant,
wherein the modifying comprises introducing a mutation at chromosome 2 position 16835660 comprising a G by T single nucleotide mutation in cDNA position 595 resulting in amino acid change from aspartic acid to tyrosine at expressed protein position 199, and/or a mutation at chromosome 2 position 17087862 comprising a G by T single nucleotide mutation in cDNA position 903 resulting in amino acid change from glutamine to histidine at expressed protein position 301, and
wherein the modifying confers resistance to an herbicide selected from the group consisting of clomazone, bixlozone, or a combination thereof.

20. The method of producing herbicide resistant rice of claim 19, the method comprising modifying the genome of the rice plant through use of gene editing proteins including meganucleases, TALENs, or CRISPR.
